(19) United States
(12) Patent Application Publication
(10) Pub. No.: US 2026/0232453 A1
(43) Pub. Date: Aug. 13, 2026

(54) BEARING TRAY LOCKING MECHANISM

(71) Applicant: Zimmer, Inc., Warsaw, IN (US)

(72) Inventor: Kevin Miller, Wakarusa, IN (US)

(21) Appl. No.: 19/150,841

(22) PCT Filed: Feb. 19, 2024
(86) PCT No.: PCT/US2024/016375

§ 371 Date: Jul. 24, 2025

Related U.S. Application Data
(60) Provisional application No. 63/447,030, filed on Feb. 20, 2023.

(51) Int. Cl. A61F 2/40; A61F 2/30

(52) U.S. Cl. CPC A61F 2/4014; A61F 2002/305; A61F 2002/3054; A61F 2002/4022; A61F 2002/4051

(57) ABSTRACT

An adjustable implant (100) can include an attachment device (110), a tray (120), and a bearing (130). The tray can be configured to be attached to the attachment device. The tray can include receptacles spaced around the tray. The receptacles can be formed in or defined by the tray. The bearing can be configured to be coupled to the tray. The bearing can include attachment mechanisms (132) spaced around the bearing and engageable with the receptacles to couple the bearing to the tray. Each one of the attachment mechanisms can be engageable with each one of the receptacles, in the alternative, such that a rotational position of the bearing relative to the tray can be adjusted by engaging the attachment mechanisms with different ones of the receptacles in the tray.

### CLAIM OF PRIORITY

This application claims the benefit of U.S. Provisional Patent Application Ser. No. 63/447,030, filed on Feb. 20, 2023, the benefit of priority of which is claimed hereby, and which is incorporated by reference herein in its entirety.

### TECHNICAL FIELD

Embodiments described herein generally relate to an implant and, more specifically, to an implant with a bearing tray locking mechanism.

### BACKGROUND

The shoulder joint is a complex joint with the scapula, clavicle, and humerus all coming together to enable a wide range of movement, at least in a properly functioning joint. In a properly functioning shoulder joint, the head of the humerus fits into a shallow socket in the scapula, typically referred to as the glenoid. Articulation of the shoulder joint involves movement of the humeral head in the glenoid, with the structure of the mating surfaces and surrounding tissues providing a wide range of motion.

The shoulder joint can undergo degenerative changes caused by various issues, such as rheumatoid arthritis, osteoarthritis, rotator cuff arthroplasty, vascular necrosis, or bone fracture. When severe joint damage occurs, and no other means of treatment can be found to be effective, a total, partial, or reverse shoulder replacement or reconstruction may be necessary.

### BRIEF DESCRIPTION OF THE DRAWINGS

Various examples are illustrated in the figures of the accompanying drawings. Such examples are demonstrative and not intended to be exhaustive or exclusive embodiments of the present subject matter.

FIG. 1 illustrates a side view of an implant, according to an example of the present disclosure.

FIG. 2 illustrates a perspective view of a tray, according to an example of the present disclosure.

FIG. 3 illustrates a side view of a tray, according to an example of the present disclosure.

FIG. 4 illustrates a partial cross-sectional view of an implant, according to an example of the present disclosure.

FIG. 5 illustrates an enlarged view of a portion of a partial cross-sectional view of an implant, according to an example of the present disclosure.

FIG. 6 illustrates an enlarged view of a portion of an implant, according to an example of the present disclosure.

FIG. 7 illustrates a bottom view of a bearing, according to an example of the present disclosure.

FIG. 8 illustrates a partial view of a portion of a bearing, according to an example of the present disclosure.

FIG. 9 illustrates an enlarged view of a portion of an implant, according to an example of the present disclosure.

FIG. 10 illustrates a bottom view of an example of a bearing, according to an example of the present disclosure.

FIG. 11 illustrates a side view of an example of a bearing, according to an example of the present disclosure.

FIG. 12 illustrates a bottom view of an example of a bearing, according to an example of the present disclosure.

FIG. 13 illustrates a side view of an example of a bearing, according to an example of the present disclosure.

FIG. 14 illustrates a side view of another example of an implant, according to an example of the present disclosure.

FIG. 15 illustrates a side view of another example of an implant, according to an example of the present disclosure.

FIG. 16 illustrates a perspective view of a tray, according to an example of the present disclosure.

FIG. 17 illustrates a side view of a tray, according to an example of the present disclosure.

FIG. 18 illustrates a perspective view of bearing, according to an example of the present disclosure.

FIG. 19 illustrates a perspective view of an underside of a bearing, according to an example of the present disclosure.

FIG. 20 illustrates a cross-sectional view of a portion of an implant, according to an example of the present disclosure.

FIG. 21 illustrates a side view of another example of an implant, according to an example of the present disclosure.

FIG. 22 illustrates a perspective view of a tray, according to an example of the present disclosure.

FIG. 23 illustrates a perspective view of an underside of a bearing, according to an example of the present disclosure.

FIG. 24 illustrates a cross-sectional view of a portion of an implant, according to an example of the present disclosure.

### DETAILED DESCRIPTION

The inventors of the present disclosure have recognized a need for a low-profile implant that can be adjusted before or during the implantation into a patient. The present disclosure describes an implant that can be adjusted. The implant can include a tray and a bearing component. The tray can be attachable to an attachment device, such as a humeral stem or a glenoid baseplate, which can be implantable into the bone of the patient. The tray can include a receptacles spaced around the tray. The receptacles can be formed in the tray such that the receptacles extend through the tray. The bearing can be configured to be coupled to the tray. The bearing can include an attachment mechanisms. The attachment mechanisms can be spaced around the bearing and can be engageable with the receptacles of the tray.

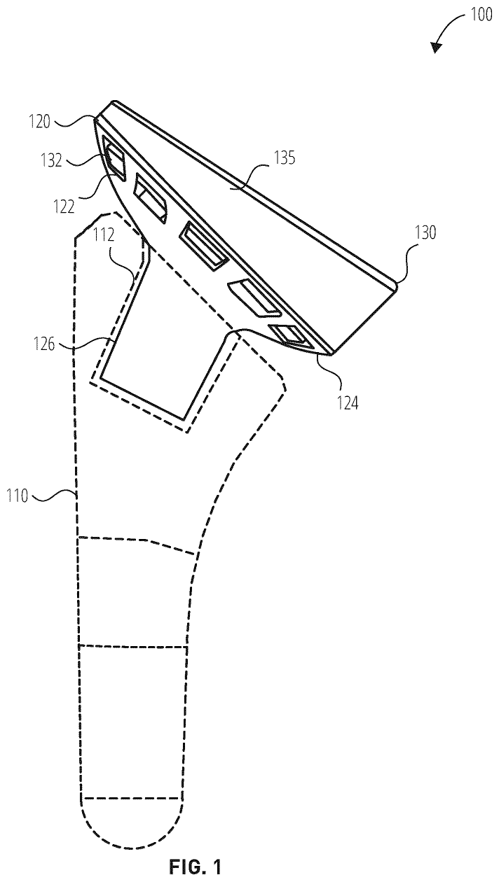

Each one of the attachment mechanisms can be engageable with each one of the receptacles, in the alternative, such that a rotational position of the bearing relative to the tray can be adjusted by engaging the attachment mechanisms with different ones of the receptacles in the tray. The adjustable implant can also include different attachment mechanisms that can help secure or provide support for the bearing attached to the tray. The adjustable implant, including a bearing tray locking mechanism, will be discussed below with reference to FIG. 1 to FIG. 24.

FIG. 1 illustrates an adjustable implant 100, according to an example of the present disclosure. The adjustable implant 100 can be inserted into a body of a patient. For example, the adjustable implant 100 can be implanted into a shoulder of a patient during a shoulder replacement or reconstruction. The adjustable implant 100 can include an attachment device 110, a tray 120, and a bearing 130. The tray 120 can include a receptacles (receptacles 122). The bearing 130 can also include an attachment mechanisms (attachment mechanisms 132).

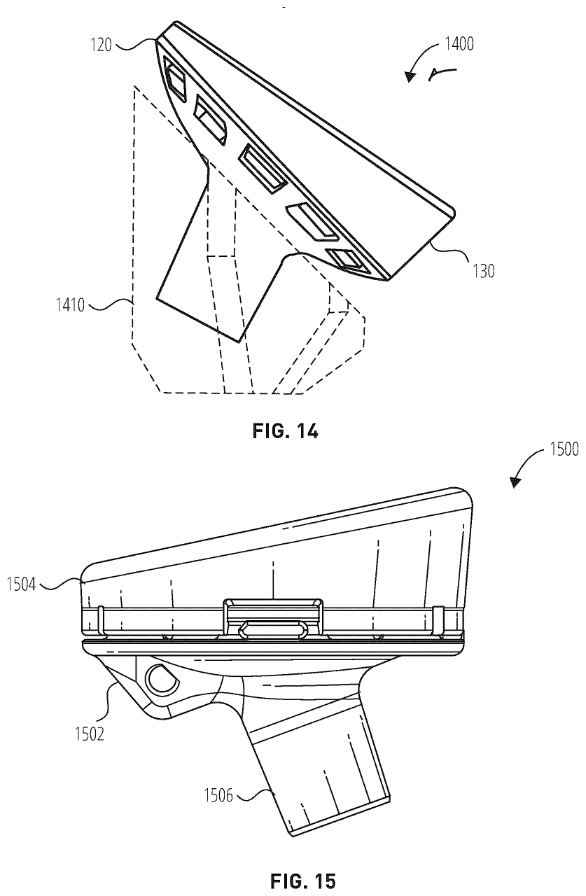

The attachment device 110 can be configured to be inserted within a bone of the patient. As shown in FIG. 1, the attachment device 110 can be a stem that can be inserted into a reamed or broached portion of the bone of a patient. In another example, the attachment device 110 can be a stemless attachment mechanism that can be inserted into a reamed or broached portion of a bone. The stemless example of the attachment device 110 is discussed below with reference to FIG. 14. The attachment device 110 can help transfer the forces from the adjustable implant 100 to the bone and other anatomy (e.g., tendons, ligaments, muscles, or the like) of the patient. In examples, the attachment device 110 can include titanium, ceramic, stainless steel, any other biocompatible material, any alloy, composite, or any combination thereof, or the like.

The tray 120 can be configured to be attached to the attachment device 110. The tray can, for example, extend into the attachment device 110. The tray 120 can attach to the attachment device 110 using common attachment strategies known in the field of implants. For example, the tray 120 can be impacted onto the attachment device 110 to couple the tray 120 to the attachment device 110, such as via a press fit connection, a friction fit connection, a taper lock connection, or the like. In one example, the attachment device 110 can include a receptacle 112 and the tray 120 can include a tapered male portion 126. As such, the receptacle 112 can be complementary to the tapered male portion 126 such that the tapered male portion 126 fits within the receptacle 112. In another example, the attachment device 110 can include a tapered male portion and the tray 120 can include a tapered receptacle that is complementary to the tapered male portion of the attachment device 110 such that the tapered receptacle of the tray 120 can receive the tapered male portion of the attachment device 110 therewithin. The tray 120 can include a peripheral region 124. In examples, the tray 120 can include titanium, cobalt-chromium, stainless steel, any combination or alloy thereof, or the like.

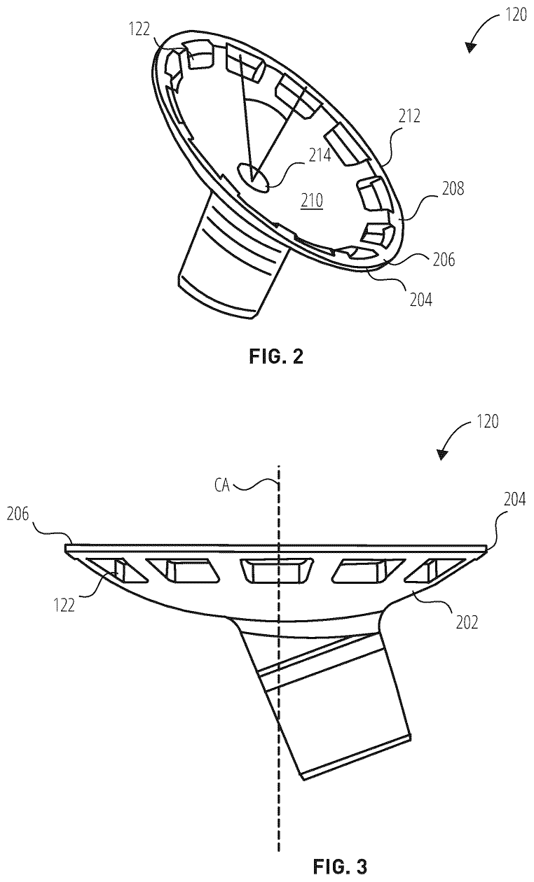

The receptacles 122 can be formed in the tray 120 such that the receptacles 122 extend through the tray 120. Because the receptacles 122 extend through the tray 120 the overall thickness of the tray 120 can remain thinner and still permit assembly of the tray 120 and the bearing 130. As shown in FIG. 1, the receptacles 122 can be circumferentially spaced around the peripheral region 124 of the tray 120. The receptacles 122 can be in the peripheral region 124 because the peripheral region 124 can be exposed to less stress than other regions of the tray 120. In another example, the receptacles 122 can be formed in any portion of the tray 120. For example, the receptacles 122 can be formed near or around a central region of the tray 120. In yet another example, the receptacles 122 can be staggered, such that the receptacles 122 alternate between being formed near the peripheral region 124 and near the central region of the tray 120. The tray 120 and the receptacles 122 will be discussed in more detail with reference to FIG. 2 to FIG. 24.

The bearing 130 can be configured to be coupled to the tray 120. The bearing 130 can also be configured to engage with another component of a shoulder, for example, a humeral head or a glenoid sphere. The bearing 130 can include a peripheral region 134 (first shown in FIG. 7) and a periphery 135. The bearing 130 can include polyethylene, ceramic, any combination or composite thereof, or the like. In examples, the tray 120 and the bearing 130 can be made in a plurality of sizes such that a surgeon can choose a tray and a bearing that will optimize the outcome for the patient. As such, the receptacles 122 and the attachment mechanisms 132 can be scaled for all sizes of the tray 120 and the bearing 130.

The attachment mechanisms 132 can be circumferentially spaced around the peripheral region 134 of the bearing 130. The attachment mechanisms 132 can be engageable with the receptacles 122 of the tray 120 to couple the bearing 130 to the tray 120. Each attachment mechanism of the attachment mechanisms 132 can be engageable with each one of the receptacles 122, in the alternative, such that a rotational position of the bearing 130 relative to the tray 120 can be adjusted by engaging the attachment mechanisms 132 with different ones of the receptacles 122 in the tray 120. The tray 120 and the bearing 130 will be discussed in more detail herein with reference to FIG. 2 to FIG. 24.

FIG. 2 illustrates a perspective view of a tray 120, according to an example of the present disclosure. FIG. 3 illustrates a side view of a tray 120, according to an example of the present disclosure. FIG. 2 and FIG. 3 will be discussed together below. In an example, the tray 120 can include a first curved surface 202 (first shown in FIG. 3), a planar surface 206, and a second curved surface 210.

The first curved surface 202 can be configured to face the attachment device 110 when the tray 120 is coupled to the attachment device 110. The first curved surface 202 can have a curvature to help increase a clearance between the tray 120 and the attachment device 110 so that the taper of the tray seats within the attachment device without impingement when the tray 120 is coupled to the attachment device 110. The first curved surface 202 can also help minimize an amount of bone of a patient that is removed to prepare the bone for implantation of the implant. In one example, the clearance between the attachment device 110 and the tray 120 can be around 0.4 mm. In another example, the clearance between the attachment device 110 and the tray 120 can be between 0.2 and 0.7 mm. In yet another example, the clearance between the attachment device 110 and the tray 120 can be between 0.1 and 0.9 mm.

The planar surface 206 can be configured to face away from the attachment device 110 when the tray 120 is coupled to the attachment device 110. The planar surface 206 can extend around a periphery 204 of the tray 120. The planar surface 206 can be configured to support the bearing 130 around a periphery of the bearing 130. Such support can help resist detachment of the bearing 130 from the tray 120. Further, the planar surface 206 can help ensure that bearing 130 is appropriately installed on the tray 120 because if bearing 130 is not seated correctly on the tray 120, there would be a gap between the planar surface 206 and the bearing 130.

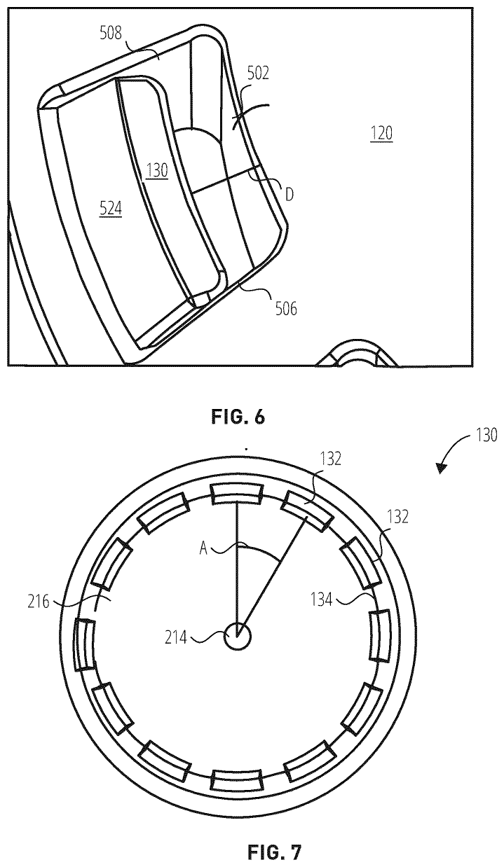

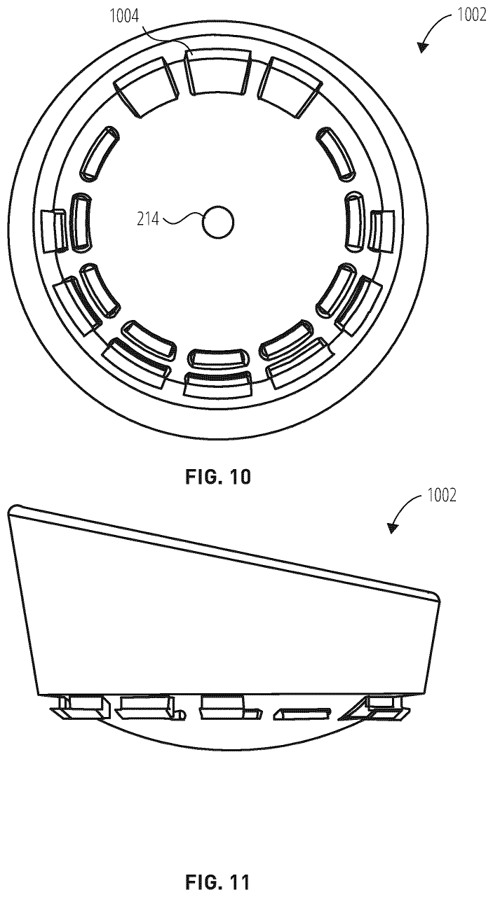

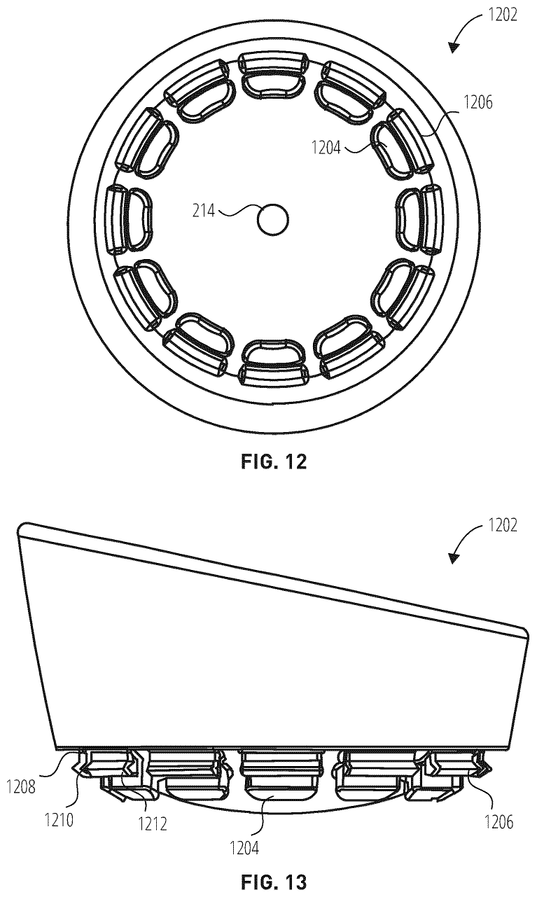

The second curved surface 210 can be configured to face away from the attachment device 110 when the tray 120 is coupled to the attachment device 110. The second curved surface 210 can extend inward from the planar surface 206. For example, the second curved surface 210 can face the bearing 130 such that the second curved surface 210 can engage the bearing 130 when the bearing 130 is attached to the tray 120. As shown in FIG. 2, the second curved surface 210 can include a flat portion 214. The flat portion 214 can be aligned with a central axis CA (first shown in FIG. 3) of the second curved surface 210. In another example, the flat portion 214 can be offset from the central axis CA of the tray 120. The flat portion 214 can provide further support for the bearing 130 when the bearing 130 is attached to the tray 120. As shown in FIG. 6, FIG. 10, and FIG. 12, the bearing 130 can include a complementary flat portion to help seat the bearing on the tray 120. Further, the flat portion 214 and the flat portion on the bearing 130 can help reduce the overall assembled height of the tray 120 and the bearing 130.

As shown in FIG. 2 and FIG. 3, the receptacles 122 can be circumferentially spaced around a middlemost portion 208 of the planar surface 206 and a peripheral portion 212 of the second curved surface 210. In another example, the receptacles 122 can be moved toward or away from the periphery 204 of the tray 120 such that the receptacles 122 do not begin at the middlemost portion 208 of the planar surface 206. The receptacles 122 can extend from the first curved surface 202 through the planar surface 206 and the second curved surface 210.

In examples, the receptacles 122 can be spaced around the tray 120 such that there can be a 30-degree spacing between each of the receptacles 122. For example, the receptacles 122 can be spaced around the tray 120 such that there can be a 20-40 degree spacing between each of the receptacles 122. In examples, the receptacles 122 can be spaced around the tray 120 such that there can be 2-40 degree spacing between each of the receptacles 122. In another example, the receptacles 122 can be spaced around the tray 120 such that there can be 1-50 degree spacing between each of the receptacles 122. In another example, the receptacles 122 can be spaced around the tray 120 at any circumferential spacing that can allow for variation in the adjustments of the bearing 130 and the tray 120 and can provide adequate support against lift off or lever out, rotation, or translation of the bearing 130 relative to the tray 120.

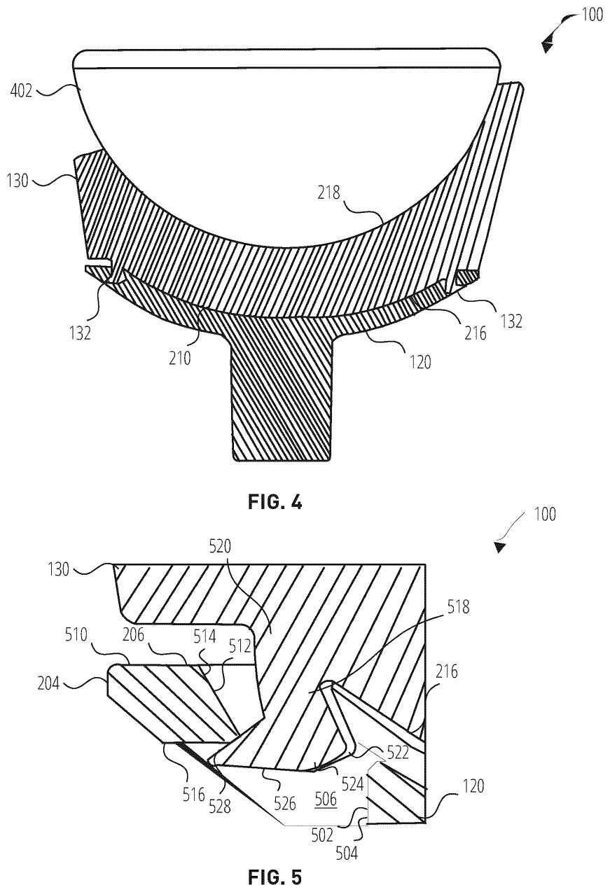

FIG. 4 illustrates a partial cross-sectional view of an adjustable implant 100, according to an example of the present disclosure. In an example, the bearing 130 can include a tray mating surface 216 and an articular surface 218.

The articular surface 218 can be configured to face away from the tray 120. The articular surface 218 can be engageable and can be operable with a second implantable component 402. For example, the glenosphere or the second implantable component 402 and the articular surface 218 can mimic the humerus head and the glenoid cavity in the scapula of a patient, respectively. As discussed above, the second implantable component 402 can be a glenoid sphere component installed to replace a humeral head of a patient. In examples, the adjustable implant 100 can be used in reverse shoulder replacements such that the humerus head or ball is moved from the humerus to the glenoid side of the joint.

The tray mating surface 216 can be configured to face the second curved surface 210 of the tray 120 when the bearing is coupled to the tray 120. As discussed above and shown in FIG. 6, FIG. 10, and FIG. 12, the tray mating surface 216 can include a flat portion that is complementary with the flat portion 214 (first shown in FIG. 2) of the second curved surface 210. The tray mating surface 216 can have a contour, curvature, or other shapes that match and is complementary with the second curved surface 210 such that the tray mating surface 216 abuts the second curved surface 210 of the tray 120 when the bearing 130 is attached to the tray 120. Such a contour of the tray mating surface 216 can encourage a smooth transfer of the bearing loads into the tray 120.

The tray mating surface 216 can include the attachment mechanisms 132. For example, the attachment mechanisms 132 can be formed on the tray mating surface 216 such that the attachment mechanisms 132 can extend from the tray mating surface 216 in a direction away from the articular surface 218.

FIG. 5 illustrates an enlarged view of a portion of a partial cross-sectional view of the adjustable implant 100, according to an example of the present disclosure. At least one of the receptacles 122 can include a first edge 502, a second edge 506, a third edge 508 (first shown in FIG. 6), and a locking tab 510. The locking tab 510 can include an inclined surface 514 and a planar surface 516. At least one of the attachment mechanisms 132 can include a body 518 and an attachment tab 524. The body 518 can include a base 520 and a distal portion 522. The attachment tab 524 can include a tapered surface 526 and a planar surface 528.

The first edge 502 can extend circumferentially and define a middlemost portion 504 of the receptacles 122. The second edge 506 can extend from the first edge 502 toward the periphery 204 of the tray 120. The third edge 508 can extend from the first edge 502 opposite from the second edge 506 and toward the periphery 204 of the tray 120.

The locking tab 510 can extend between the second edge 506 and the third edge 508 opposite from the first edge 502. The locking tab 510 can define a peripheral edge 512 of the receptacles 122. The locking tab 510 can be formed in the tray 120 to help hold the bearing 130 onto the tray 120 after the bearing 130 is attached to the tray 120. For example, the locking tab 510 can help prevent lift off, or lever out, of the bearing 130 from the tray 120. The locking tab 510 can also engage with the second edge 506 or the third edge 508 to provide support to the bearing 130 and the tray 120 against translation or rotation of the bearing 130 relative to the tray 120. In an example, the locking tab 510 can include an inclined surface 514 and a planar surface 516.

The inclined surface 514 can extend from the planar surface 226 toward the first curved surface 202. The inclined surface 514 can be inclined at any angle relative to the planar surface 206 of the tray 120. For example, the planar surface 206 can be adjusted to increase or decrease a bending of the attachment tab 524 to alter a resistance of attachment between the tray 120 and the bearing 130. For example, the angle can be increased to around 90 degrees to minimize the resistance of attachment between the tray 120 and the bearing 130 generated by the locking tab 510. In another example, the angle can be decreased to around 0 degrees to minimize the resistance of attachment between the tray 120 and the bearing 130 generated by the locking tab 510. The resistance of attachment can be adjusted to provide feedback to an operator. For example, as the bearing 130 gets closer to being attached to the tray 120, the resistance of attachment can be higher than the resistance of attachment at any other point of the attachment of the bearing 130 and the tray 120.

The planar surface 516 of the locking tab 510 can extend from the first curved surface 202 towards the first edge 502 of the respective receptacle 122. The planar surface 516 can engage with the attachment tab 524 when the attachment tab 524 is installed within the receptacles 122 to attach the bearing 130 to the tray 120. As shown in FIG. 4 and FIG. 5, the planar surface 516 can be essentially parallel to the planar surface 206. In another example, the planar surface 516 can be angled such that an edge of the planar surface 516 nearest the periphery 204 of the tray 120 is closer to the planar surface 206 than an edge of the planar surface 516 farthest the periphery 204 of the tray 120. In another example, the planar surface 516 can be angled such that the edge of the planar surface 516 nearest the periphery 204 of the tray 120 is farther from the planar surface 206 than an edge of the planar surface 516 farthest the periphery 204 of the tray 120. The planar surface 516 can engage with the attachment tab 524 to help hold the attachment tab 524 within the receptacles 122.

The body 518 can extend radially from the tray mating surface 216 between the base 520 and the distal portion 522. The attachment tab 524 can extend from the distal portion 522 of the body 518 towards the periphery 135 of the bearing 130. The tapered surface 526 of the attachment tab 524 can face away from the bearing 130. The tapered surface 526 can taper as the attachment tab 524 extends from the body 518 toward the periphery 135 of the bearing 130. The tapered surface 526 can help reduce a clearance needed between the first edge 502 and the locking tab 510 such that the locking tab 510 can have enough room to bend as the bearing 130 is being attached to the tray 120 without contacting the first edge 502. Moreover, the tapered surface 526 can also help guide the locking tab 510 into the respective one of the receptacles 122 as the bearing 130 is being attached to the tray 120.

The planar surface 528 can face toward the bearing 130 and can extend from the body 518 toward the periphery 135 of the bearing 130. The planar surface 528 can engage with the tray 120, and more specifically, with the planar surface 516 of the tray 120 to hold the bearing 130 and the tray 120 together once the bearing 130 has been attached to the tray 120. The planar surface 528 of the attachment tab can be configured to be adjacent the planar surface 528 of the locking tab 510 when the bearing can be coupled to the tray 120. In another example, the planar surface 528 can be angled such that an edge of the planar surface 528 nearest the periphery 135 of the bearing 130 is closer to the tapered surface 526 than an edge of the planar surface 528 farthest from the periphery 135 of the bearing 130. In another example, the planar surface 528 can be angled such that the edge of the planar surface 528 nearest the periphery 135 of the bearing 130 is farther from the tapered surface 526 than an edge of the planar surface 528 farthest from the periphery 135 of the bearing 130.

In an example, the tapered surface 526 of the attachment tab 524 can be configured to contact the inclined surface 514 of the locking tab 510 and bend the body 518 away from the periphery 135 of the bearing 130 as the bearing 130 is being attached to the tray 120. As discussed herein, the bending of the body 518 can generate resistance to attaching the bearing 130 to the tray 120, which can provide feedback to an operator that they are getting closer to attaching the bearing 130 to the tray 120.

FIG. 6 illustrates an enlarged view of a portion of an adjustable implant 100, according to an example of the present disclosure. The attachment mechanisms 132 can return to an un-bent position once the attachment tab 524 of the bearing 130 clears the locking tab 510 of the receptacles 122.

A distance D can be a distance between the first edge 502 of the receptacles 122 and the body 518 (FIG. 5) of the attachment mechanisms 132. The distance D can be altered by either changing a thickness of the body 518 of the attachment mechanisms 132 or changing a distance between the first edge 502 and the locking tab 510 (FIG. 2) of the receptacles 122. The distance D can be optimized. For example, the smaller the distance D, the less material removed from the tray 120, the stronger the tray 120 and the thicker the body 518 can be. Thus, the stronger the attachment mechanisms 132. However, the distance D needs to permit the attachment mechanisms 132 to bend during installation of the bearing 130 onto the tray 120 without the attachment mechanisms 132 contacting the first edge 502 of the receptacles 122. For example, the distance D can have a minimum value of 1 mm. In another example, the distance D can have a minimum value between 0.5 mm and 2 mm. Moreover, the shorter the body 518 and the shallower the attachment tab 524, the less distance D can be required to allow the attachment mechanism 132 to bend during the attachment of the bearing 130 to the tray 120.

FIG. 7 illustrates a bottom view of a bearing, according to an example of the present disclosure. As shown in FIG. 6, the attachment mechanisms 132 can be the same. Further, the attachment mechanisms 132 can have even circumferential spacing around a peripheral region of the bearing 130. The attachment mechanisms 132 can be spaced about the bearing 130 at angle A. In examples, the spacing of the attachment mechanisms 132 at angle A can be complementary to the spacing of the receptacles 122 on the tray 120 (as shown in FIG. 2 and FIG. 3). For example, if the receptacles 122 on the tray 120 are spaced apart from each other such that there is a 30-degree spacing between each other, the attachment mechanisms 132 can also have a 30-degree spacing between each other so that the attachment mechanisms 132 are complementary to the receptacles 122. Similar to the receptacles 122 of the tray 120, the spacing of the attachment mechanisms 132 of the bearing 130 can be altered to change the variability of the positioning of the bearing 130 relative to the tray 120.

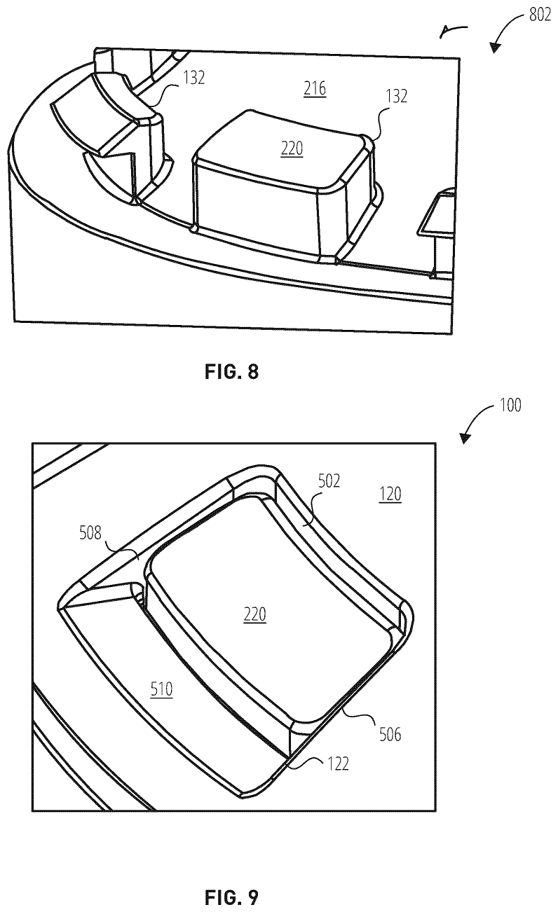

FIG. 8 illustrates a partial view of a portion of a bearing 802, according to an example of the present disclosure. The bearing 802 (e.g., the bearing 130) can be attached to the tray 120 (FIG. 2).

In an example, at least one of the attachment mechanisms 132 can be a lug 220. The lug 220 can extend radially from the tray mating surface 216. The lug 220 can be shaped complementary to at least one of the receptacles 122. The bearing 802 and the lug 220 can be one monolithic piece. In another example, the lug 220 can be coupled, attached, or fixed to the bearing 802. Though the lug 220 does not engage with the locking tab 510 (FIG. 5) to prevent lift off, or lever out, of the bearing 802 and the tray 120, the lug 220 can provide support against torsional loads. For example, because the lug 220 can be wider than other of the attachment mechanisms 132, the lug 220 can withstand greater torsional loads before failing. The lug 220 and the interactions with the receptacles 122 of the tray 120 will be discussed in more detail below with reference to FIG. 9.

FIG. 9 illustrates an enlarged view of a portion of an adjustable implant 100, according to an example of the present disclosure. As shown in FIG. 9, the bearing 802 can be attached to the tray 120 such that the lug 220 is within one of the receptacles 122 of the tray 120. For example, the lug 220 can fit within at least one of the receptacles 122 such that, when installed within the receptacles 122, the lug 220 can contact at least one of the first edge 502, the second edge 506, the third edge 508, or the locking tab 510 of the receptacles 122 to prevent movement of the bearing with relation to the tray 120. In examples, when the lug 220 is installed in the receptacles 122, the lug 220 can contact all of the first edge 502, the second edge 506, the third edge 508, and the locking tab 510. As the lug 220 can fill most of the attachment mechanisms 132, the lug 220 can engage with any of the first edge 502, the second edge 506, the third edge 508, or the locking tab 510 to provide support against torsional forces. As such, the lug 220 can also help prevent unintended translation of the bearing 802 with relation to the tray 120 after the bearing 802 has been attached to the tray 120.

FIG. 10 and FIG. 11 will be discussed together below. FIG. 10 illustrates a bottom view of a bearing 330, according to an example of the present disclosure. FIG. 11 illustrates a side view of the bearing 330, according to an example of the present disclosure.

In an example, at least one of the attachment mechanisms 132 can be a toe-in attachment mechanism 340. The toe-in attachment mechanism 1004 can be configured to engage with the attachment tab 524 (FIG. 5) of the receptacles 122 (FIG. 2) and to fit within the receptacles 122. For example, the toe-in attachment mechanism 340 can fit within the receptacles 122 such that the attachment tab 524 contacts at least one of the first edge 502 (FIG. 5), the second edge 506 (FIG. 5), the third edge 508 (FIG. 6), or the locking tab 510 (FIG. 5). The contact between the toe-in attachment mechanism 340 and at least one of the first edge 502, the second edge 506, the third edge 508, and the locking tab 510 can help prevent movement of the bearing 330 relative to the tray 120.

For example, the contact between the toe-in attachment mechanism 1004 and the locking tab 510 can help prevent a lift off or a lever out of the bearing 1002 and the tray 120. In examples, the toe-in attachment mechanism 1004 can be installed on the lateral side to better resist lever-out forces applied in a medial direction. Moreover, because of the larger size of the toe-in attachment mechanism 1004, as compared to the attachment mechanisms 132 of FIG. 5, the toe-in attachment mechanism 1004 can provide more support against torsional forces and can engage with either the second edge 506 or the third edge 508 to prevent rotation or translation of the bearing 1002 relative to the tray 120. In an example, the toe-in attachment mechanism 1004 can be configured to be inserted into the receptacles 122 before any other of the attachment mechanisms 132.

FIG. 12 and FIG. 13 will be discussed together. FIG. 12 illustrates a bottom view of a bearing 1202, according to an example of the present disclosure. FIG. 13 illustrates a side view of the bearing 1202, according to an example of the present disclosure. The bearing 1202 can be attached to the tray 120 (FIG. 2). As shown in FIG. 12 and FIG. 13, the attachment mechanisms 132 of the bearing 1202 can include a lug portion 1204 and an attachment portion 1206.

The lug portion 1204 can be positioned radially inward from the attachment portion 1206 such that the lug portion 1204 can engage with any one of the first edge 502 (FIG. 5), the second edge 506 (FIG. 5), or the third edge 508 (FIG. 6) of the receptacles 122. The lug portion 1204 can fill a portion of the space between the first edge 502 and the second edge 506 to provide support for the bearing 1202 and the tray 120 against torsional forces. For example, the lug portion 1204 can engage with any of the first edge 502, the second edge 506, or the third edge 508 to prevent translation or rotation of the bearing 1202 relative to the tray 120.

The attachment portion 1206 can be positioned radially outward from the lug portion 1204 such that the attachment portion 1206 can engage with any one of the second edge 506, the third edge 508, or the locking tab 510 (FIG. 5). Similar to the attachment mechanisms 132 discussed in FIG. 5 5, the attachment portion 1206 can include a base 1208, a locking tab 1210, and an inclined surface 1212.

The base 1208 can extend from the tray mating surface 216 of the attachment portion 1206 can extend between a base and a distal portion. The locking tab 1210 can extend from the base 1208 from the distal portion. The locking tab 1210 can be configured to engage with the attachment tab 524 of the receptacles 122 (as discussed in FIG. 5). The inclined surface 1212 can help provide clearance between the attachment portion 1206 and the lug portion 1204 while the bearing 1202 is being attached to the tray 120.

The attachment portion 1206, and more specifically, the locking tab 1210 of the attachment portion 1206, can help hold the bearing 1202 and the tray 120 together after the bearing 1202 has been attached to the tray 120. For example, the locking tab 1210 can engage with the attachment tab 524 of the tray 120 to help resist lifting off the bearing 1202 from the tray 120. The attachment portion 1206 can also engage with the second edge 506 and the third edge 508 to provide support to the bearing 1202 and the tray 120 against rotation or translation.

The lug portion 1204 can be spaced from the attachment portion 1206 such that the attachment portion 1206 can bend radially inward toward the lug portion 1204, but without contacting the lug portion 1204, while the bearing 1202 is being attached to the tray 120. The spacing between the attachment portion 1206 reduces the material required to make the bearing 1202 and provides both support from detachment and translation or rotation of the bearing 1202 relative to the tray 120.

The bearings shown in FIG. 1 and FIG. 4-F IG. 12, for example, the bearing 130, the bearing 802, the bearing 1002, and the bearing 1202, are illustrative examples of bearings that can be used with the tray 120 to make an adjustable implant that has a low clearance between the tray and the attachment device 110 (FIG. 1). Any combination thereof the bearings shown in FIG. 1 and FIG. 4-FIG. 12, for example, the bearing 130, the bearing 802, the bearing 1002, and the bearing 1202 can be implemented to adjust various parameters of the adjustable implant 100. For example, more of the attachment mechanisms 132 can include the lug 220 to help prevent translation or rotation of the bearing relative to the tray, or all of the attachment mechanisms 132 can include the lug portion 1204 and the lug portion 1204 to improve resistance to lift off, or lever out, while also improving the resistance to translation or rotation of the bearing relative to the tray.

FIG. 14 illustrates a side view of an adjustable implant 1400, according to an example of the present disclosure. As shown in FIG. 14, the attachment device 1410 (e.g., the attachment device 110 from FIG. 1) can be a stemless attachment device. As such, the implant 1400 can include the tray 120 and any of the bearings shown in FIGS. 1, 4-12, for example, the bearing 130, the bearing 802, the bearing 1002, and the bearing 1202. As such, the adjustable implant 1400 can include a tray and a bearing that are adjustable in relation to each other, which can include a low clearance between the bearing and the attachment device 1410.

In examples described here, the tray 120 includes receptacles and the bearing 130 includes attachment mechanisms. In another example, the relationship of the connection can be reversed. For example, the tray 120 can include attachment mechanisms that are configured to engage with receptacles of the bearing 130.

FIG. 15 illustrates a side view of another example of an adjustable implant 1500 (e.g., the adjustable implant 100 (first shown in FIG. 1) or the adjustable implant 1400 (FIG. 14)), according to an example of the present disclosure. The adjustable implant 1500 can include a different attachment interface between the tray and bearings as compared to the adjustable implant 100 or the adjustable implant 1400. For example, the adjustable implant 1500 can include an edge snap, which can couple a bearing to a tray.

Even with the different attachment interfaces, the adjustability of the adjustable implant 1500 remains similar to that of the adjustable implant 100 and the adjustable implant 1400 such that the rotational position of the bearing relative to the tray can be adjusted intra-operatively by the medical professionals completing the surgical procedure. In examples, the adjustable implant 1500 can include a tray 1502, a bearing 1504, and a male tapered portion 1506.

The tray 1502 can be configured to be attached to an attachment device (e.g., the attachment device 110 (first shown in FIG. 1) or the attachment device 1410 (FIG. 14)). For example, the male tapered portion 1506 of the tray 1502 can be configured to be inserted into the attachment device to couple the tray 1502 to the attachment device and provide support to the tray via the attachment device such as to spread out loads that the tray 1502 is exposed to after implantation into the shoulder of a patient. The tray 1502 can include titanium, cobalt-chromium, stainless steel, any combination or alloy thereof, or the like.

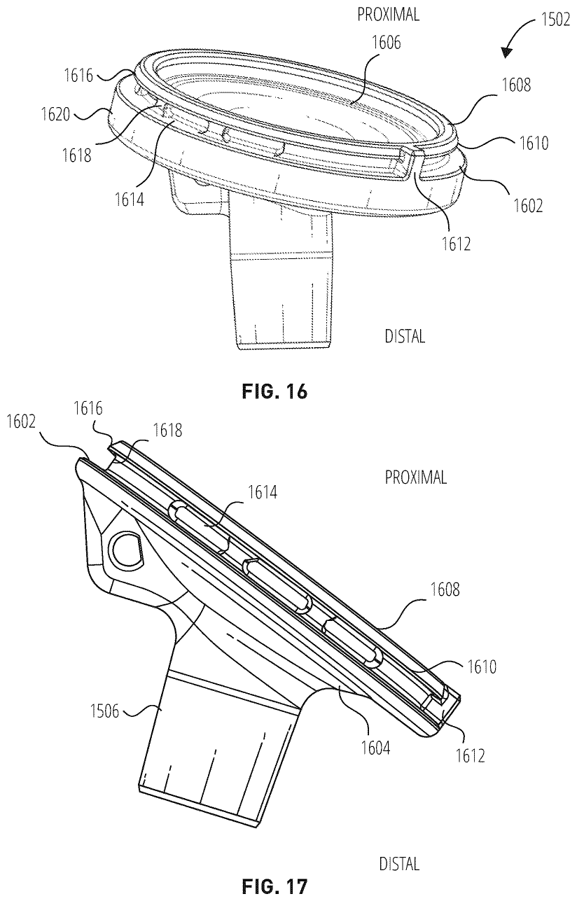

The bearing 1504 can be configured to be coupled to the tray 1502. The bearing 1504 can also be configured to engage with another component of a shoulder, for example, a humeral head or a glenoid sphere. The bearing 1504 can include polyethylene, ceramic, any combination or composite thereof, or the like. The tray 1502 and the bearing 1504 can be made in a plurality of sizes such that a surgeon can choose a tray and a bearing that will optimize the outcome for the patient. The tray 1502, the male tapered portion 1506, and the bearing 1504 will be discussed in more detail herein with reference to FIG. 16-FIG. 20.

FIG. 16 and FIG. 17 will be discussed together herein. FIG. 16 illustrates a perspective view of the tray 1502 (e.g., the tray 120 (FIG. 1)), according to an example of the present disclosure. FIG. 17 illustrates a side view of an example of the tray 1502, according to an example of the present disclosure. The tray 1502 can include a planar surface 1602, a first curved surface 1604 (first shown in FIG. 17), a second curved surface 1606, a locking ridge 1608, a locking barb 1610, an alignment boss 1612, and a receptacles 1614 extending from a radially outer surface 1616 and a radially inner surface 1618.

The planar surface 1602 can be configured to face away from the attachment device when the tray 1502 is coupled to the attachment device. The planar surface 1602 can extend around a periphery 1620 of the tray 1502. The planar surface 1602 can be configured to support the bearing 1504 around a periphery of the bearing 1504. Such support can help resist the detachment of the bearing 1504 from the tray 1502. Further, the planar surface 1602 can help ensure that bearing 1504 is appropriately installed on the tray 1502 because if bearing 1504 is not seated correctly on the tray 1502, there can be a gap between the planar surface 1602 and the bearing 1504.

The first curved surface 1604 can be configured to face the attachment device when the tray 1502 is coupled to the attachment device. The first curved surface 1604 can include a curvature to help increase a clearance between the tray 1502 and the attachment device so that the taper (e.g., the male tapered portion 1506) of the tray 1502 seats within the attachment device without impingement when the tray 1502 is coupled to the attachment device. The first curved surface 1604 can also help minimize the amount of bone that is removed to prepare the bone for implantation of the implant. In one example, the clearance between the attachment device and the tray 1502 can be around 0.4 mm. In another example, the clearance between the attachment device and the tray 1502 can be between 0.2 and 0.7 mm. In yet another example, the clearance between the attachment device and the tray 1502 can be between 0.1 and 0.9 mm.

The second curved surface 1606 can be configured to face away from the attachment device when the tray 1502 is coupled to the attachment device. The second curved surface 1606 can extend inward from the planar surface 1602. For example, the second curved surface 1606 can face the bearing 1504 such that the second curved surface 1606 can engage the bearing 1504 when the bearing 1504 is attached to the tray 1502.

The locking ridge 1608, which can be a part of the attachment receptacles, can extend upward from the planar surface 1602 and extend circumferentially around the planar surface 1602. The locking ridge 1608 can extend around an entire circumference of the planar surface 1602. In examples, the locking ridge 1608 can extend around just a portion of the circumference of the planar surface 1602. As shown in FIG. 16, the locking ridge 1608 can be radially offset from the periphery 1620 of the tray 1502 such that the locking ridge 1608 is radially inward the periphery 1620 of the tray 1502. Such an offset of the locking ridge 1608 can vary around the circumference of the tray 1502 such that a radial inward offset of the locking ridge 1608 can vary around the periphery 1620 of the tray 1502. The locking ridge 1608 can be engageable with the bearing 1504 to couple the bearing 1504 to the tray 1502. The locking ridge 1608 can include the locking barb 1610.

The locking barb 1610 can be configured to engage with the bearing 1504 to retain the bearing 1504 onto the tray 1502 after the bearing 1504 is coupled to the tray 1502. The locking barb 1610 can extend radially outward from a proximal portion of the locking ridge 1608. The locking barb 1610 can also extend toward the periphery 1620 of the tray 1502.

The attachment receptacles (e.g., the receptacles 1614) of the tray 1502 can also include the alignment boss 1612, which can extend upward (e.g., proximally) from the planar surface 1602. The alignment boss 1612 can also extend radially outward from the locking ridge 1608 and to the periphery 1620 of the tray 1502. The alignment boss 1612 can be configured to engage with a portion of the bearing 1504 to set a rotational position of the bearing 1504 relative to the tray 1502 at known increments.

As shown in FIG. 16, the receptacles 1614 can be defined by the locking ridge 1608 and the locking barb 1610. For example, the locking barb 1610 can form the receptacles 1614 circumferentially around locking ridge 1608 such that the receptacles 1614 can extend from the radially outer surface 1616 of the locking ridge 1608 to the radially inner surface 1618 of the locking ridge 1608. In other words, the receptacles 1614, which are configured to receive the bearing 1504, can extend around an entire circumference of the tray 1502 as defined by the locking ridge 1608 and the locking barb 1610.

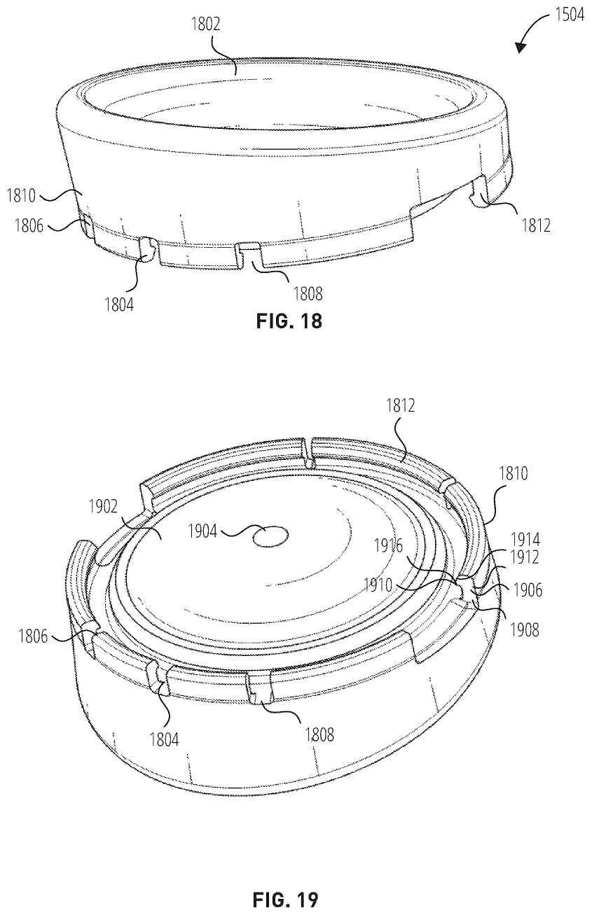

FIG. 18 and FIG. 19 will be discussed together below. FIG. 18 illustrates a perspective view of bearing 1504 (e.g., the bearing 130, the bearing 802, the bearing 1002, the bearing 1202), according to an example of the present disclosure. FIG. 19 illustrates a perspective view of an underside of the bearing 1504, according to an example of the present disclosure. The bearing 1504 can be configured to engage with the tray 1502 to couple to the tray 1502 and be rotationally adjustable relative to the tray 1502. The bearing 1504 can include an articular surface 1802, a first alignment relief notch 1804, a second alignment relief notch 1806, a third alignment relief notch 1808, a portion of a perimeter 1810, and an edge protrusion 1812 (e.g., attachment mechanisms 132).

The first alignment relief notch 1804 can be configured to receive the alignment boss 1612 of the tray 1502 to define the rotational position of the bearing 1504 relative to the tray 1502. In examples, the first alignment relief notch 1804 can be formed in the edge protrusion 1812. The first alignment relief notch 1804 can extend proximally into the bearing 1504. In examples, the first alignment relief notch 1804 can also extend radially through at least a portion of the bearing 1504 toward the articular surface 1802. As shown in FIG. 18, the first alignment relief notch 1804 can extend all the way through the edge protrusion 1812 to the tray mating surface 1902.

The second alignment relief notch 1806 and the third alignment relief notch 1808 can also be formed in the edge protrusion 1812. The second alignment relief notch 1806 and the third alignment relief notch 1808 can also be formed in the bearing 1504. In examples, the second alignment relief notch 1806 can be circumferentially spaced from the first alignment relief notch 1804 in a first direction and the third alignment relief notch 1808 can be circumferentially spaced from the first alignment relief notch 1804 in a second direction, which can be opposite the first direction.

The alignment boss 1612 can be insertable, in the alternative, into any one of the first alignment relief notch 1804, the second alignment relief notch 1806, or the third alignment relief notch 1808 to define the rotational position of the bearing 1504 relative to the tray 1502 as defined by the first alignment relief notch 1804, the second alignment relief notch 1806, and the third alignment relief notch 1808, respectively.

As shown in FIG. 19, the bearing 1504 can include a tray mating surface 1902 (e.g., the tray mating surface 216 (first shown in FIG. 7), and a center 1904. The tray mating surface 1902 can be configured to engage with the second curved surface 1606 of the tray 1502 when the bearing 1504 is coupled to the tray 1502.

The edge protrusion 1812 can be configured to attach the bearing 1504 to the tray 1502. The edge protrusion 1812 can define at least a portion of a perimeter 1810 of the bearing 1504. The edge protrusion 1812 can extend around a periphery of the bearing 1504 to engage with the tray 1502. As shown in FIG. 18 and FIG. 19, the edge protrusion 1812 can extend from the perimeter of the bearing 1504 and toward the center 1904 of the bearing 1504. The edge protrusion 1812 can be configured to engage with the tray 1502 to couple the bearing 1504 to the tray 1502 and determine a rotational position of the bearing 1504 relative to the tray 1502.

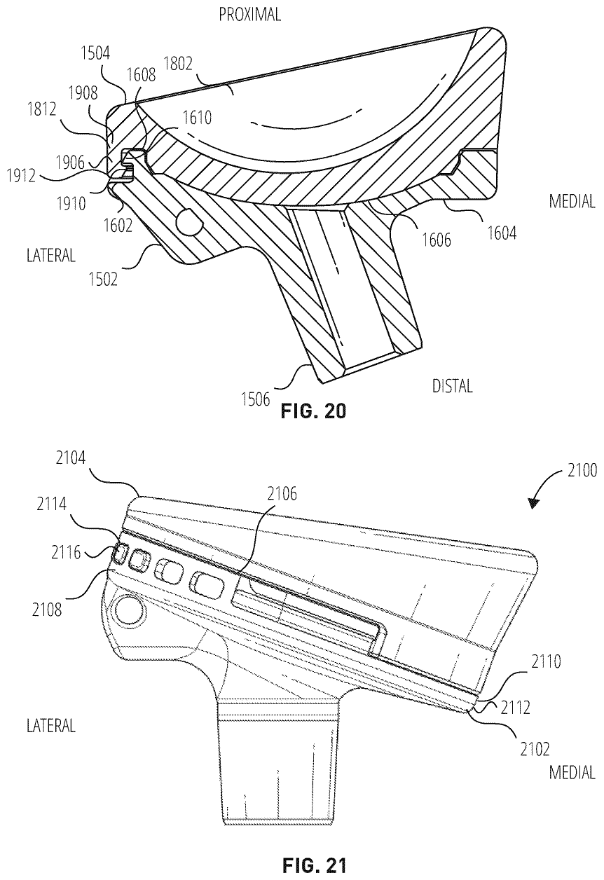

As such, the bearing 1504, and more specifically, the edge protrusion 1812, can include a body 1906, which can extend from the bearing 1504 at a base 1908 and toward a distal portion of the body 1912. The edge protrusion 1812 can also include an attachment tab 1910. The attachment tab 1910 can include a tapered surface 1914 (e.g., tapered surface 526) and a planar surface 1916 (e.g., planar surface 528). The tapered surface 526 can face away from the bearing 1504 and can taper as the attachment tab (e.g., the attachment tab 1910 (FIG. 20)) extends toward the center 1904 of the bearing 1504.

The planar surface 1916 can face the bearing 1504 and can extend from the body (e.g., body 1906 (FIG. 20)) toward the center 1904 of the bearing 1504. The adjustable implant 1500 will also be discussed in FIG. 20.

FIG. 20 illustrates a cross-sectional view of a portion of the adjustable implant 1500, according to an example of the present disclosure. As best shown in FIG. 20, when the bearing 1504 is coupled to the tray 1502, the planar surface 1916 of the attachment tab 1910 can be configured to engage a distal surface of the locking barb 1610. Thus, during the installation of the bearing 1504 onto the tray 1502, the attachment tab 1910 can contact the locking barb 1610 and bend the body 1906 away from the center 1904 of the bearing 1504 as the bearing 1504 is being attached to the tray 1502. Once the bearing 1504 is installed onto the tray 1502, the body 1906 can bend back toward the center 1904 such that the planar surface 1916 of the attachment tab 1910 is located adjacent to a distal edge of the locking barb 1610. Although the locking ridge 1608 and the locking barb 1610 of the tray 1502 and the attachment tab 1910 of the bearing 1504 are only shown on the lateral portions of the tray 1502 and the bearing 1504, respectively, the locking ridge 1608 and the locking barb 1610 of the tray 1502 and the attachment tab 1910 of the bearing 1504 can circumferentially extend around the tray 1502 and the bearing 1504, respectively, such that they are also on the medial portions of the tray 1502 and the bearing 1504.

In summary, the medical professional can intra-operatively adjust a rotational position of the bearing 1504 relative to the tray 1502 by aligning the alignment boss 1612 of the tray 1502 with the preferred alignment notch (e.g., the first alignment relief notch 1804, the second alignment relief notch 1806, or the third alignment relief notch 1808) while coupling the bearing 1504 to the tray 1502.

FIG. 21 illustrates a side view of another example of an adjustable implant 2100 (e.g., the adjustable implant 100 (first shown in FIG. 1) the adjustable implant 1400 (FIG. 14), or the adjustable implant 1500 (first shown in FIG. 15)), according to an example of the present disclosure. The adjustable implant 2100 can include a different attachment interface between the tray and bearings as compared to the adjustable implant 100, the adjustable implant 1400, or the adjustable implant 1500. For example, the adjustable implant 2100 can include a ledge snap, which can couple a bearing to a tray.

Even with the different attachment interfaces, the adjustability of the adjustable implant 1500 remains similar to that of the adjustable implant 100, the adjustable implant 1400, and the adjustable implant 1500, such that the rotational position of the bearing relative to the tray can be adjusted intra-operatively by the medical professionals completing the surgical procedure.

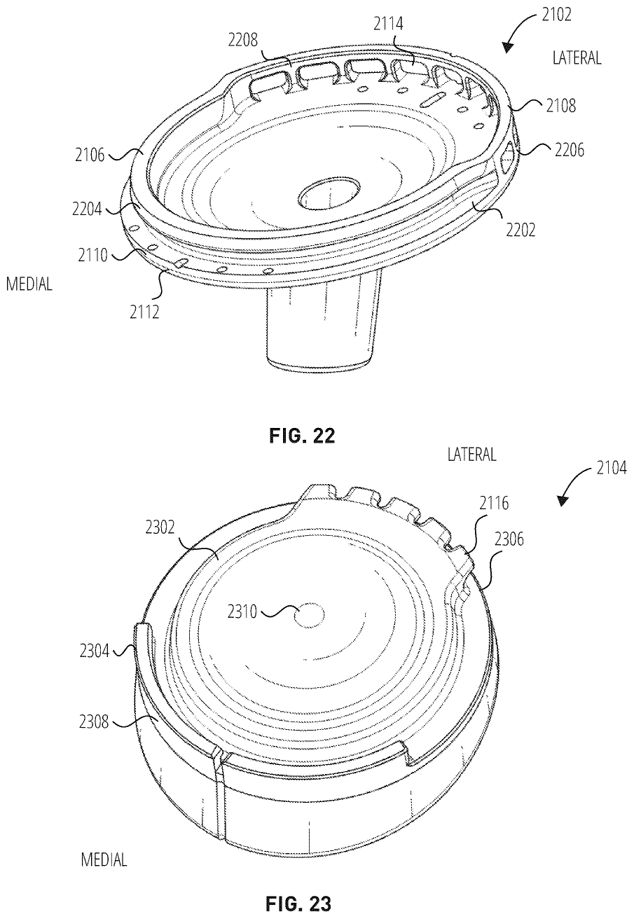

The adjustable implant 2100 can include a tray 2102 (e.g., the tray 120 (first shown in FIG. 1) or the tray 1502 (first shown in FIG. 15)), and a bearing 2104 (e.g., bearing 130 (first shown in FIG. 1), the bearing 802 (first shown in FIG. 8), the bearing 1002 (first shown in FIG. 10), the bearing 1202 (first shown in FIG. 12), or the bearing 1504 (first shown in FIG. 15)). The tray 2102 can include a locking ridge 2106 extending from a planar surface (e.g., planar surface 2202 (first shown in FIG. 22). The locking ridge 2106 can include lateral portion 2108 and a medial portion 2110 (each of the lateral portion 2108 and the medial portion 2110 extending around at least a portion of the perimeter 2112). The locking ridge 2106 can include receptacles 2114 formed in the lateral portion 2108 of the locking ridge 2106 and a locking barb 2204 (first shown in FIG. 22) in the medial portion 2110 of the locking ridge 2106. The bearing 2104 can include a lug 2116 configured to be inserted within the receptacles 2114 in the lateral portion 2108 of the locking ridge 2106 and an edge protrusion 2304 (first shown in FIG. 23) configured to attach to the locking ridge 2106 and the locking barb 2204 in the medial portion 2110 of the locking ridge 2106.

FIG. 22 illustrates a perspective view of an example of the tray 2102 (e.g., the tray 120 (first shown in FIG. 1) or the tray 1502 (first shown in FIG. 15)), according to an example of the present disclosure. The tray 2102 can include a planar surface 2202 extending around at least a portion of the perimeter 2112 of the tray 2102. The tray 2102 can include attachment receptacles, which can include a locking ridge 2106 that extends upward from the planar surface 2202 and circumferentially around the planar surface 2202. As discussed above, the locking ridge 2106 can include a medial portion 2110 and a lateral portion 2108. In the medial portion 2110 of locking ridge 2106, the attachment receptacles can include a locking barb 2204, and in the lateral portion 2108 of the locking ridge 2106 the attachment receptacles can include receptacles 2114, which can extend through the locking ridge 2106 from a radially outer surface 2206 and through a radially inner surface 2208.

The medial portion 2110 of the locking ridge 2106 can be offset radially within the perimeter 2112 of the tray 2102. In the medial portion 2110 of the locking ridge 2106, the amount of radial offset can change as the locking ridge 2106 extends circumferentially around the tray 2102. For example, the offset of the locking ridge 2106 from the perimeter 2112 can be minimized at the most medial portion of the medial portion 2110 and can increase as the locking ridge 2106 extends away from the most medial portion of the medial portion 2110 of the tray 2102.

The locking barb 2204 can extend from a distal edge of the locking ridge 2106 toward the perimeter 2112 of the medial portion 2110. The locking barb 2204 can extend the entire length of the medial portion 2110 of the locking ridge 2106. The locking barb 2204 can extend at least a portion of the length of medial portion 2110 of the locking ridge 2106. The extension of the locking barb 2204 toward the perimeter 2112 of the tray 2102 can change as the locking barb 2204 extends circumferentially along the locking ridge 2106. For example, the locking barb 2204 can extend farther from the locking ridge 2106 toward the medial edge and can extend less from the locking ridge 2106 as it moves away from the medial portion edge. In another example, the locking barb 2204 can have a maximum extension from the locking ridge 2106 within the medial portion 2110 of the tray 2102.

In the lateral portion 2108 of the locking ridge 2106, the locking ridge 2106 can define the perimeter 2112 of the tray 2102. The receptacles 2114 formed in the lateral portion 2108 of the locking ridge 2106 can extend from a radially outer surface 2206 and through a radially inner surface 2208 of the lateral portion 2108 of the tray 2102. The receptacles 2114 can be circumferentially spaced along the lateral portion 2108 of the locking ridge 2106. As will be discussed herein, the locking ridge 2106 and the locking barb 2204 can be configured to engage with the edge protrusion of the bearing 2104, and the receptacles 2114 can be configured to receive the lugs 2116 of the bearing 2104.

FIG. 23 illustrates a perspective view of an underside of an example of the bearing 2104, according to an example of the present disclosure. The bearing 2104 can include a flat spot at a center 2310 of the tray mating surface 2302. Also formed in the tray mating surface 2302 are the attachment mechanisms (e.g., the attachment mechanisms 132 (FIG. 1)). As shown in FIG. 23, the on the lateral portion of the bearing 2104, the attachment mechanisms can include at least one lug 2116 and on the medial portion of the bearing 2104, the attachment mechanisms can include at least one edge protrusion 2304.

The lug 2116 can extend radially outward from the center 2310 of the bearing 2104 and toward a lateral perimeter 2306 of the bearing 2104. The lug 2116 can be shaped complementary to at least one of the attachment receptacles (e.g., the receptacles 2114 (FIG. 22)) formed in the lateral portion 2108 of the locking ridge 2106 such that the lug 2116 can fit within at least one of the receptacles 2114 to prevent movement of the bearing 2104 relative to the tray 2102. The lug 2116 can engage with the receptacles 2114 to prevent both rotational and axial movements of the bearing 2104 relative to the tray 2102.

The edge protrusion 2304 can be formed in the bearing 2104 radially outside of the tray mating surface 2302 such that the edge protrusion 2304 forms at least a portion of a medial perimeter 2308 of the bearing 2104. The edge protrusion 2304 can be configured to engage with the locking ridge 2106 and the locking barb 2204 of the tray 2102 to couple the tray 2102 to the bearing 2104. The edge protrusion 2304 can define at least a portion of a medial perimeter 2308.

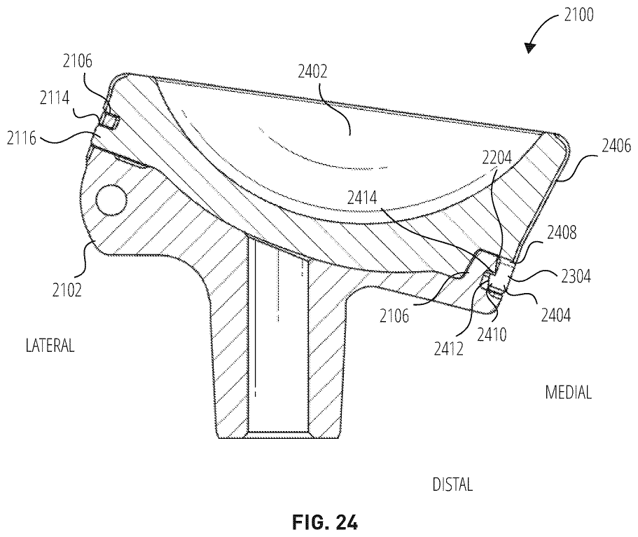

FIG. 24 illustrates a cross-sectional view of a portion of an example of the adjustable implant 2100 (e.g., the adjustable implant 100 (FIG. 1), the adjustable implant 1400 (FIG. 14), the adjustable implant 1500 (FIG. 15)), according to an example of the present disclosure. As first shown in FIG. 24, the bearing 2104 can include an articular surface 2402 (e.g., articular surface 218 (FIG. 2)).

On the lateral side of the tray 2102 and the bearing 2104 the receptacles 2114 formed in the locking ridge 2106 of the tray 2102 can receive the lug 2116 of the attachment mechanism of the bearing 2104. As discussed herein, the engagement between the receptacles 2114 and the lug 2116 can help hold the rotational position of the bearing 2104 relative to the tray 2102 and translation (e.g., proximal movement) of the bearing 2104 relative to the tray 2102.

On the medial side of the tray 2102 and the bearing 2104 the locking ridge 2106 and the locking barb 2204 of the tray 2102 can be configured to engage with the edge protrusions 2304 of the bearing 2104. As discussed herein, the engagement between the locking ridge 2106 and the locking barb 2204 of the tray 2102 and the edge protrusion 2304 of the bearing 2104 can prevent translation (e.g., proximal movement) of the bearing 2104 relative to the tray 2102.

The edge protrusion 1812 can include a body 2404. The body 2404 can at least partially define a perimeter 2406 of the bearing 2104. The body 2404 can include a base 2408 extending adjacent to the tray mating surface 2302. The body 2404 can also include an attachment tab 2410 (e.g., the attachment tab 524 (FIG. 5) or the attachment tab 1910 (FIG. 19)). The attachment tab 2410 can extend from a distal portion of the body 2404 toward the center 2310 (shown in FIG. 23) of the bearing 2104. The attachment tab 2410 can include a taper tapered surface 2412 and a planar surface 2414. The tapered surface 2412 can be configured to face away from the bearing 2104. The tapered surface 2412 can taper as the attachment tab 2410 extends from the body 2404 toward the center 2310 of the bearing 2104. The planar surface 2414 can be configured to face toward the bearing 2104 and extend from the body 2404 toward the center 2310 of the bearing 2104.

In examples, the tapered surface 2412 of the attachment tab 2410 can be configured to contact the locking barb 2204 and bend the body 2404 away from the center 2310 of the bearing 2104 as the bearing 2104 is being attached to the tray 2102. The planar surface 2414 of the attachment tab 2410 can be configured to engage a distal surface of the locking barb 2204 when the bearing 2104 is coupled to the tray 2102. As such, the planar surface 2414 of the attachment tab 2410 can engage with the locking ridge 2106 and the locking barb 2204 of the tray 2102 to hold the bearing 2104 and the tray 2102 together after the bearing 2104 is coupled to the tray 2102.

In summary, the medical professional can intraoperatively adjust the rotational position of the bearing 2104 relative to the tray 2102 by inserting the lug 2116 (or lugs 2116) into the receptacles 2114 correlating to the preferred rotational position of the bearing 2104 relative to the tray 2102.

The following, non-limiting examples, detail certain aspects of the present subject matter to solve the challenges and provide the benefits discussed herein, among others. Example 1 is an adjustable implant comprising: an attachment device implantable into a bone of a patient; a tray configured to be attached to the attachment device, the tray including: a receptacles circumferentially spaced around a peripheral region of the tray, the receptacles formed in the tray such that the receptacles extend through the tray; and a bearing configured to be coupled to the tray, the bearing including: attachment mechanisms circumferentially spaced around a peripheral portion of the bearing and engageable with the receptacles of the tray to couple the bearing to the tray, each one of the attachment mechanisms engageable with each one of the receptacles, in the alternative, such that a rotational position of the bearing relative to the tray can be adjusted by engaging the attachment mechanisms with different ones of the receptacles in the tray.

In Example 2, the subject matter of Example 1 optionally includes wherein the attachment device is a stem.

In Example 3, the subject matter of any one or more of Examples 1-2 optionally include wherein the tray comprises: a first curved surface configured to face the attachment device when the tray is coupled to the attachment device; a planar surface configured to face away from the attachment device when the tray is coupled to the attachment device, the planar surface extending around a periphery of the tray; and a second curved surface configured to face away from the attachment device when the tray is coupled to the attachment device, the second curved surface extending inward from the planar surface.

In Example 4, the subject matter of Example 3 optionally includes wherein the attachment receptacles comprise: a locking ridge extending upward from the planar surface and circumferentially around the planar surface, the locking ridge including: a locking barb extending radially outward from a proximal portion of the locking ridge toward a perimeter of the tray; and an alignment boss extending upward from the planar surface and from the locking ridge to the perimeter of the tray.

In Example 5, the subject matter of Example 4 optionally includes wherein the locking barb extends circumferentially around locking ridge and extending radially inward from a radial outward surface of the locking ridge to a radial inward surface of the locking ridge.

In Example 6, the subject matter of Example 5 optionally includes wherein the bearing comprises: a tray mating surface configured to face the second curved surface of the tray when the bearing is coupled to the tray; and an articular surface configured to face away from the tray.

In Example 7, the subject matter of Example 6 optionally includes wherein the attachment mechanisms comprise: an edge protrusion formed in the bearing radially outside of the tray mating surface such that the edge protrusion form at least a portion of a perimeter of the bearing.

In Example 8, the subject matter of Example 7 optionally includes wherein edge protrusion comprises: a body at least partially defining the perimeter of the bearing, the body including: a base extending adjacent to the tray mating surface; and an attachment tab extending from a distal portion of the body toward a center of the bearing.

In Example 9, the subject matter of Example 8 optionally includes wherein the attachment tab comprises: a tapered surface facing away from the bearing, the tapered surface tapers as attachment tab extends from the body toward the center of the bearing; and a planar surface facing towards the bearing and extending from the body toward the center of the bearing.

In Example 10, the subject matter of Example 9 optionally includes wherein the tapered surface of the attachment tab is configured to contact the locking barb and bend the body away from the center of the bearing as the bearing is being attached to the tray, and wherein the planar surface of the attachment tab is configured to engage a distal surface of the locking barb when the bearing is coupled to the tray.

In Example 11, the subject matter of any one or more of Examples 6-10 optionally include wherein the bearing comprises: a first alignment relief notch formed in the attachment mechanisms and configured to receive the alignment boss of the tray to define the rotational position of the bearing relative to the tray.

In Example 12, the subject matter of Example 11 optionally includes wherein the bearing comprises: a second alignment relief notch formed in the attachment mechanisms and circumferentially spaced from the first alignment relief notch in a first direction; and a third alignment relief notch formed in the attachment mechanisms and circumferentially spaced from the first alignment relief notch in a second direction opposite the first direction.

In Example 13, the subject matter of Example 12 optionally includes wherein the alignment boss boss is insertable, in the alternative, into any one of the first alignment relief notch, the second alignment relief notch, or the third alignment relief notch to define the rotational position of the bearing relative to the tray as defined by the first alignment relief notch, the second alignment relief notch, and the third alignment relief notch, respectively.

In Example 14, the subject matter of any one or more of Examples 3 -13 optionally include wherein the attachment receptacles comprise: a locking ridge extending upward from the planar surface and circumferentially around the planar surface, the locking ridge including: a lateral portion extending circumferentially around at least a portion of a lateral edge of the tray, the lateral portion defining at least a portion of a perimeter of the tray; and a medial portion extending circumferentially around at least a portion of a medial edge of the tray, the medial portion offset radially inward from the perimeter of the tray.

In Example 15, the subject matter of Example 14 optionally includes wherein in the lateral portion of the locking ridge the attachment receptacles extend through the locking ridge from a radially outer surface of the locking ridge to a radially inner surface of the locking ridge.

In Example 16, the subject matter of Example 15 optionally includes wherein in the medial portion of the locking ridge, the attachment receptacles comprise: a locking barb extending radially outward from a distal portion of the locking ridge toward the perimeter of the tray.

In Example 17, the subject matter of Example 16 optionally includes wherein the bearing comprises: a tray mating surface configured to face the second curved surface of the tray when the bearing is coupled to the tray; and an articular surface configured to face away from the tray, the articular surface engageable with a second implantable component.

In Example 18, the subject matter of Example 17 optionally includes wherein the attachment mechanisms comprises: a lug extending radially outward from the tray mating surface and toward a lateral perimeter of the bearing, the lug shaped complementary to at least one of the attachment receptacles formed in the lateral portion of the locking ridge such that the lug can fit within at least one of the receptacles to prevent movement of the bearing relative to the tray; and an edge protrusion formed in the bearing radially outside of the tray mating surface such that the edge protrusion forms at least a portion of a medial perimeter of the bearing.

In Example 19, the subject matter of Example 18 optionally includes wherein edge protrusion comprises: a body at least partially defining the perimeter of the bearing, the body including: a base extending adjacent to the tray mating surface; and an attachment tab extending from a distal portion of the body toward a center of the bearing.

In Example 20, the subject matter of Example 19 optionally includes wherein the attachment tab comprises: a tapered surface facing away from the bearing, the tapered surface tapers as attachment tab extends from the body toward the center of the bearing; and a planar surface facing towards the bearing and extending from the body toward the center of the bearing.

In Example 21, the subject matter of Example 20 optionally includes wherein the tapered surface of the attachment tab is configured to contact the locking barb and bend the body away from the center of the bearing as the bearing is being attached to the tray, and wherein the planar surface of the attachment tab is configured to engage a distal surface of the locking barb when the bearing is coupled to the tray.

In Example 22, the subject matter of any one or more of Examples 3-21 optionally include wherein the receptacles are circumferentially spaced around a middlemost portion of the planar surface and a peripheral portion of the second curved surface, and wherein the receptacles extend from the first curved surface and through the planar surface and the second curved surface.

In Example 23, the subject matter of Example 22 optionally includes wherein the bearing comprises: a tray mating surface configured to face the second curved surface of the tray when the bearing is coupled to the tray; and an articular surface configured to face away from the tray, the articular surface engageable with a second implantable component.

In Example 24, the subject matter of Example 23 optionally includes wherein at least one of the receptacles comprises: a first edge extending circumferentially and defining a middlemost portion of the receptacle; a second edge extending from the first edge toward the periphery of the tray; a third edge extending from the first edge opposite the second edge and toward the periphery of the tray; and a locking tab extending between the second edge and the third edge opposite the first edge and defining a peripheral edge of the receptacle.

In Example 25, the subject matter of Example 24 optionally includes wherein the locking tab includes: an inclined surface tapering from the planar surface toward the first curved surface; and a planar surface essentially parallel to the planar surface, the planar surface of the locking tab extending from the first curved surface towards the first edge of the respective receptacle.

In Example 26, the subject matter of Example 25 optionally includes wherein at least one of the attachment mechanisms comprises: a body including: a base attached to the tray mating surface; and a distal portion, the body extending radially from the tray mating surface between the base and the distal portion; and an attachment tab extending from the distal portion of the body towards a periphery of the bearing.

In Example 27, the subject matter of Example 26 optionally includes wherein the attachment tab comprises: a tapered surface facing away from the bearing, the tapered surface tapers as attachment tab extends from the body toward the periphery of the bearing; and a planar surface facing towards the bearing and extending from the body toward the periphery of the bearing.

In Example 28, the subject matter of Example 27 optionally includes wherein the tapered surface of the attachment tab is configured to contact the inclined surface of the locking tab and bend the body away from the periphery of the bearing as the bearing is being attached to the tray, and wherein the planar surface of the attachment tab is configured to be adjacent the planar surface of the locking tab when the bearing is coupled to the tray.

In Example 29, the subject matter of Example 28 optionally includes wherein at least one of the attachment mechanisms comprises: a toe-in attachment mechanism configured to engage with the attachment tab of the receptacles and fit within the receptacles and contact at least one of the first edge, the second edge, the third edge, or the locking tab to prevent movement of the bearing with relation to the tray.

In Example 30, the subject matter of Example 29 optionally includes wherein the toe-in attachment mechanism is configured to be inserted into the receptacles before any other of the attachment mechanisms.

In Example 31, the subject matter of any one or more of Examples 24-30 optionally include wherein at least one of the attachment mechanisms comprises: a lug extending radially from the tray mating surface, the lug shaped complementary to at least one of the receptacles such that the lug can fit within at least one of the receptacles and contact with at least one of the first edge, the second edge, the third edge, or the locking tab to prevent movement of the bearing with relation to the tray.

In Example 32, the subject matter of Example 31 optionally includes wherein the lug and the receptacles are complementary such that the lug can fit within the receptacles and contact at least one of the first edge, the second edge, the third edge, or the locking tab to prevent movement of the bearing with relation to the tray.

Example 33 is an adjustable implant comprising: an attachment device implantable into a bone of a patient; a tray configured to be attached to the attachment device, a tray including: a receptacles spaced around the tray, the receptacles formed in the tray such that the receptacles extend through the tray; and a bearing configured to be coupled to the tray, the bearing including: an attachment mechanisms spaced around the bearing and engageable with the receptacles of the tray to couple the bearing to the tray, each one of the attachment mechanisms engageable with each one of the receptacles, in the alternative, such that a rotational position of the bearing relative to the tray can be adjusted by engaging the attachment mechanisms with different ones of the receptacles in the tray.

In Example 34, the subject matter of Example 33 optionally includes wherein the attachment device is a stem.

In Example 35, the subject matter of Example 34 optionally includes wherein the tray comprises: a first curved surface configured to face the attachment device when the tray is coupled to the attachment device; a planar surface configured to face away from the attachment device when the tray is coupled to the attachment device, the planar surface extending around a periphery of the tray; and a second curved surface configured to face away from the attachment device when the tray is coupled to the attachment device, the second curved surface extending inward from the planar surface.

In Example 36, the subject matter of Example 35 optionally includes wherein the receptacles are circumferentially spaced around a middlemost portion of the planar surface and a peripheral portion of the second curved surface, and wherein the receptacles extend from the first curved surface and through the planar surface and the second curved surface.

In Example 37, the subject matter of Example 36 optionally includes wherein the bearing comprises: a tray mating surface configured to face the second curved surface of the tray when the bearing is coupled to the tray; and an articular surface configured to face away from the tray, the articular surface engageable with a second implantable component.

In Example 38, the subject matter of Example 37 optionally includes wherein at least one of the receptacles comprises: a first edge extending circumferentially and defining a middlemost portion of the receptacle; a second edge extending from the first edge toward the periphery of the tray; a third edge extending from the first edge opposite the second edge and toward the periphery of the tray; and a locking tab extending between the second edge and the third edge opposite the first edge and defining a peripheral edge of the receptacle.

Example 39 is at least one machine-readable medium including instructions that, when executed by processing circuitry, cause the processing circuitry to perform operations to implement any element of any of Examples 1-38.

Example 40 is an apparatus comprising means to implement any element of any of Examples 1-38.

Example 41 is a system to implement any element of any of Examples 1-38.

Example 42 is a method to implement any element of any of Examples 1-38.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used.

Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. An adjustable implant comprising:
an attachment device implantable into a bone of a patient;
a tray configured to be attached to the attachment device, the tray including:
receptacles circumferentially spaced around a peripheral region of the tray, the receptacles formed in the tray such that the receptacles extend through the tray, and
a bearing configured to be coupled to the tray, the bearing including:
attachment mechanisms circumferentially spaced around a peripheral portion of the bearing and engageable with the receptacles of the tray to couple the bearing to the tray, each one of the attachment mechanisms engageable with each one of the receptacles, in the alternative, such that a rotational position of the bearing relative to the tray can be adjusted by engaging the attachment mechanisms with different ones of the receptacles in the tray.

2. The adjustable implant of claim 1, wherein the attachment device is a stem.

3. The adjustable implant of claim 1, wherein the tray comprises:
a first curved surface configured to face the attachment device when the tray is coupled to the attachment device;
a planar surface configured to face away from the attachment device when the tray is coupled to the attachment device, the planar surface extending around a periphery of the tray; and
a second curved surface configured to face away from the attachment device when the tray is coupled to the attachment device, the second curved surface extending inward from the planar surface.

4. The adjustable implant of claim 3, wherein the receptacles comprise:
a locking ridge extending upward from the planar surface and circumferentially around the planar surface, the locking ridge including:
a locking barb extending radially outward from a proximal portion of the locking ridge toward a perimeter of the tray, wherein the locking barb extends circumferentially around the locking ridge and extends radially inward from a radial outward surface of the locking ridge to a radial inward surface of the locking ridge; and
an alignment boss extending upward from the planar surface and from the locking ridge to the perimeter of the tray.

5. (canceled)

6. The adjustable implant of claim 4, wherein the bearing comprises:
a tray mating surface configured to face the second curved surface of the tray when the bearing is coupled to the tray; and
an articular surface configured to face away from the tray;
wherein the attachment mechanisms comprise:
an edge protrusion formed in the bearing radially outside of the tray mating surface such that the edge protrusion form at least a portion of a perimeter of the bearing, and wherein the edge protrusion comprises:
a body at least partially defining the perimeter of the bearing. the body including:
a base extending adjacent to the tray mating surface; and
an attachment tab extending from a distal portion of the body toward a center of the bearing, wherein the attachment tab comprises:
a tapered surface facing away from the bearing. the tapered surface tapers as attachment tab extends from the body toward the center of the bearing: and
a planar surface facing towards the bearing and extending from the body toward the center of the bearing: and
wherein the tapered surface of the attachment tab is configured to contact the locking barb and bend the body away from the center of the bearing as the bearing is being attached to the tray, and wherein the planar surface of the attachment tab is configured to engage a distal surface of the locking barb when the bearing is coupled to the trav.

7. (canceled)

8. (canceled)

9. (canceled)

10. (canceled)

11. The adjustable implant of claim 6, wherein the bearing comprises:
a first alignment relief notch formed in the attachment mechanisms and configured to receive the alignment boss of the tray to define the rotational position of the bearing relative to the tray;
a second alignment relief notch formed in the attachment mechanisms and circumferentially spaced from the first alignment relief notch in a first direction; and
a third alignment relief notch formed in the attachment mechanisms and circumferentially spaced from the first alignment relief notch in a second direction opposite the first direction; and
wherein the alignment boss is insertable, in the alternative, into any one of the first alignment relief notch, the second alignment relief notch, or the third alignment relief notch to define the rotational position of the bearing relative to the tray as defined by the first alignment relief notch, the second alignment relief notch, and the third alignment relief notch, respectively.

12. (canceled)

13. (canceled)

14. The adjustable implant of claim 3, wherein the receptacles comprise:
a locking ridge extending upward from the planar surface and circumferentially around the planar surface, the locking ridge including:
a lateral portion extending circumferentially around at least a portion of a lateral edge of the tray, the lateral portion defining at least a portion of a perimeter of the tray; and
a medial portion extending circumferentially around at least a portion of a medial edge of the tray, the medial portion offset radially inward from the perimeter of the tray.

15. The adjustable implant of claim 14, wherein in the lateral portion of the locking ridge the receptacles extend through the locking ridge from a radially outer surface of the locking ridge to a radially inner surface of the locking ridge; and
wherein in the medial portion of the locking ridge, the receptacles comprise. a locking barb extending radially outward from a distal portion of the locking ridge toward the perimeter of the tray.

16. (canceled)

17. The adjustable implant of claim 15, wherein the bearing comprises:
a tray mating surface configured to face the second curved surface of the tray when the bearing is coupled to the tray, and
an articular surface configured to face away from the tray, the articular surface engageable with a second implantable component.

18. The adjustable implant of claim 17, wherein the attachment mechanisms comprise:
a lug extending radially outward from the tray mating surface and toward a lateral perimeter of the bearing, the lug shaped complementary to at least one of the receptacles formed in the lateral portion of the locking ridge such that the lug can fit within at least one of the receptacles to prevent movement of the bearing relative to the tray; and
an edge protrusion formed in the bearing radially outside of the tray mating surface such that the edge protrusion forms at least a portion of a medial perimeter of the bearing, wherein edge protrusion comprises:
a body at least partially defining the perimeter of the bearing, the body including:
a base extending adjacent to the tray mating surface; and
an attachment tab extending from a distal portion of the body toward a center of the bearing, wherein the attachment tab comprises:
a tapered surface facing away from the bearing, the tapered surface tapers as attachment tab extends from the body toward the center of the bearing; and
a planar surface facing towards the bearing and extending from the body toward the center of the bearing; and
wherein the tapered surface of the attachment tab is configured to contact the locking barb and bend the body away from the center of the bearing as the bearing is being attached to the tray, and wherein the planar surface of the attachment tab is configured to engage a distal surface of the locking barb when the bearing is coupled to the tray.

19. (canceled)

20. (canceled)

21. (canceled)

22. The adjustable implant of claim 3, wherein the receptacles are circumferentially spaced around a middlemost portion of the planar surface and a peripheral portion of the second curved surface, and wherein the receptacles extend from the first curved surface and through the planar surface and the second curved surface.

23. The adjustable implant of claim 22, wherein the bearing comprises:
a tray mating surface configured to face the second curved surface of the tray when the bearing is coupled to the tray; and
an articular surface configured to face away from the tray, the articular surface engageable with a second implantable component.

24. The adjustable implant of claim 23, wherein at least one of the receptacles comprises:
a first edge extending circumferentially and defining a middlemost portion of the receptacles;
a second edge extending from the first edge toward the periphery of the tray;
a third edge extending from the first edge opposite the second edge and toward the periphery of the tray, and
a locking tab extending between the second edge and the third edge opposite the first edge and defining a peripheral edge of the receptacles.

25. The adjustable implant of claim 24, wherein the locking tab includes:
an inclined surface tapering from the planar surface toward the first curved surface;
and a planar surface essentially parallel to the planar surface, the planar surface of the locking tab extending from the first curved surface towards the first edge of a respective receptacle of the receptacles.

26. The adjustable implant of claim 25, wherein at least one of the attachment mechanisms comprises:
a body including:
a base attached to the tray mating surface; and
a distal portion, the body extending radially from the tray mating surface between the base and the distal portion; and
an attachment tab extending from the distal portion of the body towards a periphery of the bearing.

27. The adjustable implant of claim 26, wherein the attachment tab comprises:
a tapered surface facing away from the bearing, the tapered surface tapers as attachment tab extends from the body toward the periphery of the bearing; and
a planar surface facing towards the bearing and extending from the body toward the periphery of the bearing.

28. The adjustable implant of claim 27, wherein the tapered surface of the attachment tab is configured to contact the inclined surface of the locking tab and bend the body away from the periphery of the bearing as the bearing is being attached to the tray, and wherein the planar surface of the attachment tab is configured to be adjacent the planar surface of the locking tab when the bearing is coupled to the tray.

29. The adjustable implant of claim 28, wherein at least one of the attachment mechanisms comprises:
a toe-in attachment mechanism configured to engage with the attachment tab of the receptacles and fit within the receptacles and contact at least one of the first edge, the second edge, the third edge, or the locking tab to prevent movement of the bearing with relation to the tray.

30. The adjustable implant of claim 29, wherein the toe-in attachment mechanism is configured to be inserted into the receptacles before any other of the attachment mechanisms.

31. The adjustable implant of claim 24, wherein at least one of the attachment mechanisms comprises:
a lug extending radially from the tray mating surface, the lug shaped complementary to at least one of the receptacles such that the lug can fit within at least one of the receptacles and contact with at least one of the first edge, the second edge, the third edge, or the locking tab to prevent movement of the bearing with relation to the tray; and
wherein the lug and the receptacles are complementary such that the lug can fit within the receptacles and contact at least one of the first edge, the second edge, the third edge, or the locking tab to prevent movement of the bearing with relation to the tray.

32. (canceled)
